(19) United States
(12) Patent Application Publication
(10) Pub. No.: US 2025/0018007 A1
(43) Pub. Date: Jan. 16, 2025

(54) SUBLINGUAL TREATMENT OF FOOD ALLERGY

(71) Applicant: STALLERGENES GREER INTERNATIONAL AG, Baar (CH)

(72) Inventors: Giampiero DE LUCA, Genéve (CH); Alessandro BERTI, Genéve (CH)

(73) Assignee: STALLERGENES GREER INTERNATIONAL AG, Baar (CH)

(21) Appl. No.: 18/711,490

(22) PCT Filed: Nov. 14, 2022
(86) PCT No.: PCT/IB2022/000812

§ 371 Date: May 17, 2024

(30) Foreign Application Priority Data
Nov. 18, 2021 (GB) 2116671.5

(51) Int. Cl. A61K 38/16; A61K 9/00; A61P 37/08

(52) U.S. Cl. CPC A61K 38/168; A61K 9/006; A61P 37/08

(57) ABSTRACT

The present invention provides therapies for use in treating a food allergy in a human patient, particular peanut protein allergy, comprising an accelerated up-dosing treatment protocol.

### FIELD

The present invention concerns food allergens for use in treating food allergy using sublingual delivery of the food allergen to the patient at least in the build-up phase of the treatment.

### BACKGROUND

Allergen Immunotherapy (AIT) comprises a build-up phase in which the allergen concentration increases gradually and a maintenance phase in which the projected dose is applied. The development of adverse reactions is associated with first dosages leading to treatment abandonment.

Preferred routes of administration for food allergens are sublingual (SLIT), oral (OIT) and/or epicutaneous (EPIT).

Food allergy is a growing public health problem with personal, social, nutritional, and economic consequences. In the United States, it is estimated that 8% of children and 10.8% of adults have food allergies. According to the FDA, about 50% of anaphylaxis reported by emergency departments is due to a food allergen. The most common triggers of severe and fatal food-induced anaphylactic reactions are peanuts and tree nuts. Allergies to peanuts are particularly worrisome as unlike allergies to other allergenic foods, such as milk and egg, which are commonly outgrown by 5 or 10 years of age, 80% of peanut allergies persist into adulthood.

The first drug for peanut allergy, Palforzia®, peanut (Arachis hypogaea) allergen powder-dnfp, an oral immunotherapy (OIT), was approved by the US Food and Drug Administration (FDA) in January 2020. Aside from Palforzia®, peanut (Arachis hypogaea) allergen powder-dnfp, however, there are no other approved treatments for food allergy, and the standard of care is strict avoidance of the specific food. Even with extreme vigilance, accidental ingestions are not uncommon, and patients remain at risk for potentially life-threatening reactions. It is estimated that approximately 40% of patients with food allergies report at least one food allergy-related emergency in their lifetime. As a result, families of children with food allergies report disruptions in daily activities, increased stress and anxiety, and lower quality of life.

OIT has been the most researched therapeutic approach for treating food allergy over the last decade. OIT and epicutaneous immunotherapy (EPIT) strategies have advanced to late-stage clinical trials. Both approaches have major drawbacks, however. OIT has limitations in terms of safety and ease of administration. Because OIT typically requires mixing peanut powder into soft foods and ingesting the food, administration is time consuming and may be inconvenient for the families of young children undergoing the OIT, leading to poor compliance and dropping out of treatment. OIT is also associated with more allergic side effects than other forms of immunotherapy, including induction of episodic anaphylaxis with dosing, dose-limiting gastrointestinal side effects in approximately 20% and eosinophilic esophagitis in less than 5% of clinical trial participants. See Burks AW′, et al., “Treatment for food allergy,” J Allergy Clin Immunol. 2018 January; 141 (1): 1-9. Dose adjustments are frequently required because of viral illness, exercise, or menses to maintain a safe dosing profile. Seasonal allergies can further complicate safety profiles and affect clinical outcomes in those treated with OIT. Id.

EPIT involves application of a small allergen patch to the back or upper arm changed at 24-hour intervals over years of therapy. EPIT for peanut allergy is associated with only modest immunologic changes to date. Id. EPIT, thus, might be limited in its ability to generate clinically meaningful immunologic changes.

Sublingual immunotherapy (SLIT) could represent a viable alternative for patients because of its simple route of administration and the good overall safety and efficacy seen in smaller trials. Four FDA-approved SLIT products for environmental allergens are commercially available, including 2 grass tablets and 1 ragweed tablet, for treating seasonal allergic rhinitis, as well as a house dust mite product (Dermatophagoides farinae and Dermatophagoides pteronyssinus combined tablet) for perennial allergic rhinitis. See Mahler V, et al., “Understanding differences in allergen immunotherapy products and practices in North America and Europe,” J Allergy Clin Immunol. 2019 March; 143 (3): 813-828. Environmental allergens, however, represent a much less dangerous allergy profile and one which is much easier to treat from a safety profile perspective. Due to the risk of anaphylaxis, treatment of environmental allergens has been considered by allergists to be non-analogous to the treatment of food such as peanut allergens.

There have been some clinical studies with sublingual therapy for treating peanut allergies, but prior protocols have been conservative and often take long periods of time for patients to be desensitized to peanut allergens, in an effort to reduce the risk of anaphylaxis. The long duration of these studies has been amongst the main cause of patient dropout. Kim et al., J Allergy Clin Immunol. 2011 March; 127 (3): 640-6.e1, Kim et al., J Allergy Clin Immunol. 2019; 144(5), 1320-1326, Orgel et al, Clin. Exp. Allergy 2019; 49:461-470, Fleischer et al., J Allergy Clin Immunol. 2013; 131(1): 119-127, Narisety et al., J Allergy Clin Immunol. 2015; 135 (5): 1275-1282e5 all disclose very long up-dosing periods, such as 26 weeks or longer, to get to the so-called maintenance dose. The prior studies have also been one-size-fits-all, subjecting patients to various long periods of up-dosing regardless of how allergic to peanuts they are. Id. No SLIT for peanut allergies has been yet FDA approved. Thus, there is room for further development of SLIT for improved treatment and prevention of food allergies, in particular, peanut allergy. We aim to show the feasibility and safety of food allergen administration during the build-up phase (also referred to as up-dosing phase) by sublingual administration in a shorter period of time compared to conventional up-dosing in AIT.

### SUMMARY

The inventors have surprisingly found that a shorter up-dosing period is safe, well tolerated and allows patients undergoing a SLIT regimen for the treatment of food allergy, particularly peanut allergy, to reach the maintenance dose in a much quicker way, thus, resulting inter alia in a better patient compliance and a reduced risk of anaphylaxis in case of accidental exposure to the food allergen.

The present invention provides the following embodiments:

1. A food allergen for use in treating a food allergy in a human patient wherein the treatment comprises an up-dosing phase followed by a maintenance phase, characterized in that the up-dosing phase is of about 60 days, optionally 45 days or further optionally 24 to 28 days and in that the food allergen is administered sublingually (also referred to as the accelerated up-dosing).

2. The food allergen for the use of embodiment 1, wherein the food allergen is administered sublingually in the maintenance phase.

3. The food allergen for the use of embodiment 1 or 2, wherein the food allergy is peanut allergy.

4. The food allergen for the use of embodiment 1, wherein the food allergen is a peanut protein

5. The food allergen for the use of any one of the preceding embodiments, wherein the treatment further comprises an initial dose escalation phase of 1 or 2 days before the up-doing phase (sometimes referred to as a rush phase).

6. The food allergen for the use of embodiment 5, wherein the initial dose escalation phase comprises administering sublingually to the patient a first dose of peanut protein of about 0.1 mg and a second dose of peanut protein of about 0.3 mg, wherein the first and the second dose are administered both on day 1 or the first dose is administered on day 1 and the second dose is administered on day 2.

7. The food allergen for the use of any one of the preceding embodiments, wherein the up-dosing phase comprises administering sublingually to the patient at least the following four different doses of peanut protein:
a) a first dose phase comprising a first dose of about 0.5 mg given daily for 5 to 7 days,
b) a second dose phase comprising a second dose of about 1.5 mg given daily for 5 to 7 days,
c) a third dose phase comprising a third dose of about 2.5 mg given daily for 5 to 7 days, and
d) a fourth dose phase comprising a fourth dose of about 4.0 mg given daily for 5 to 7 days.

8. The food allergen for the use of any one of the preceding embodiments, wherein the maintenance phase comprises administering sublingually to the patient a daily dose of peanut protein of about 4.0 mg to about 5.0 mg for at least 20 to 28 weeks, preferably for 26 weeks, and optionally for one or more additional periods (for example 1, 2, 3, 4, 5, 6, 7 or 8 periods or more) of 20 to 28 weeks each, preferably of 26 weeks, thereafter.

9. The food allergen for the use of embodiment 5, wherein the initial dose escalation phase comprises administering sublingually to the patient a first dose of peanut protein of about 0.3 mg and a second dose of peanut protein of about 0.9 mg, wherein the first and the second dose are administered both on day 1 or the first dose is administered on day 1 and the second dose is administered on day 2.

10. The food allergen for the use of any one of the preceding embodiments, wherein the up-dosing phase comprises administering sublingually to the patient at least the following four different doses of peanut protein:
a) a first dose phase comprising a first dose of about 1.2 mg given daily for 5 to 7 days,
b) a second dose phase comprising a second dose of about 2.5 mg given daily for 5 to 7 days,
c) a third dose phase comprising a third dose of about 3.5 mg given daily for 5 to 7 days, and
d) a fourth dose phase comprising a fourth dose of about 5.0 mg given daily for 5 to 7 days.

11. The food allergen for the use of any one of the preceding embodiments, wherein the maintenance phase comprises administering sublingually to the patient a daily dose of peanut protein of about 5.0 mg to about 6.0 mg for at least 20 to 28 weeks, preferably for 26 weeks, and optionally for one or more additional periods (for example 1, 2, 3, 4, 5, 6, 7 or 8 periods or more) of 20 to 28 weeks each, preferably of 26 weeks, thereafter.

12. The food allergen for the use of embodiment 5, wherein the first dose administered in the initial dose escalation phase is about 0.1% of the patient's eliciting dose (ED).

13. The food allergen for the use of any one of the preceding embodiments, wherein the patient's ED is ≤100 mg.

14. The food allergen for the use of any one of the preceding embodiments, wherein the patient's ED is 101 mg-300 mg.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### DESCRIPTION OF THE EMBODIMENTS

One or more embodiments of the invention will now be described with reference to the accompanying FIGURE, in which:

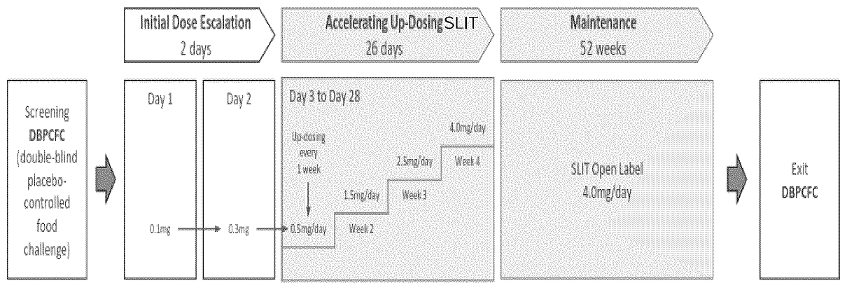

FIG. 1 shows schematically a detailed preferred embodiment of the present invention with 26 day accelerated up-dosing and incremental dosage increases. The protocol may be considered in two parts. Part A lasts 28 days and comprises the initial dose escalation (rush phase) and the accelerated up-dosing. This phase has a primary endpoint which is the safety and tolerability of SLIT accelerated up-dosing. Part B comprises a 52-week long maintenance phase; the primary endpoint is long-term safety and tolerability of peanut SLIT based on adverse events listed in Part A; the secondary endpoint is (i) the proportion of subjects who tolerate at least 300 mg of peanut protein with no more than mild symptoms at the exit DBPCFC (Double-Blind, Placebo-Controlled, Food Challenge) or 10× increase in ED (for those who had an ED less than 30 mg at screening) or (ii) the proportion of subjects who tolerate at 600 mg of peanut protein with no more than mild symptoms at the exit DBPCFC. During part B, an interim analysis can be performed at week 26.

### I. DEFINITIONS

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, disclosed antibodies are used to delay development of a disease or to slow the progression of a disease.

As used herein, “microgram” refers to one millionth of a gram (1×10⁻⁶ gram), and when used in connection with “peanut protein,” refers to micrograms of all peanut proteins combined, rather than micrograms of a particular peanut protein. Peanuts and peanut flour are about 50% protein by weight.

As used herein, “week” refers to all of the days of the week except for week 1 of the treatment protocol. For week 1 of a treatment protocol, week 1 is defined as either days 2 or 3 to 7 of week 1 (i.e., all of the days in week 1 except day 1 or day 1 and 2).

As used herein, “reflux” refers to gastroesophageal reflux or acid reflux, which occurs when stomach acid flows back into the esophagus. Reflux presents with one or more of the following symptoms: a burning sensation in the chest, usually after eating, and which might be worse at night; chest pain; difficulty swallowing; regurgitation of food or sour liquid; and/or a sensation of a lump in the throat. Reflux is significant when it requires assessment or treatment by a medical provider (for example, when it causes an amount of pain or loss of sleep that is disruptive to the patient).

As used herein the eliciting dose (ED) is the dose given during an Oral Food Challenge (OFC) that induces the onset of unequivocal allergic symptoms, or the dose that stops the challenge based on physician discretion i.e., the dose that is not tolerated. The ED is also sometime referred to as the reactive dose or the not-tolerated dose. The term “reactive dose” may therefore be considered to be the dose given during a food challenge that includes the onset of unequivocal allergic symptoms.

As used herein the “single highest tolerated dose” is the highest dose given during a food challenge that elicits no symptoms or symptoms that are not clearly indicative of an allergic reaction.

As used herein the “cumulative tolerated dose” is the sum of the tolerated doses, not including the reactive dose. It is possible to calculate the ED if the cumulative tolerated dose is known.

As used herein the “cumulative reactive dose” is the sum of doses consumed including the reactive (not-tolerated) dose.

### II. FOOD ALLERGY

Food allergy is a pathological reaction of the immune system triggered by the ingestion of a food protein antigen. Exposure to very small amounts of allergenic foods can trigger clinical symptoms such as gastrointestinal disorders, urticaria, and airway inflammation, ranging in severity from mild to life-threatening. Food allergy results from a breakdown of oral tolerance, delayed development of oral tolerance, or both in subjects genetically and possibly environmentally predisposed to atopic disease. It is distinguished from food intolerance in that intolerance does not arise from immune system dysregulation; for example, lactose intolerance arises from non-immune factors, such as lactose malabsorption and lactase deficiency

Diagnosis of food allergy is typically made through clinical history and detection of specific IgE. However, no specific therapies for food allergy are available, other than Palforzia® for peanut allergy, and the current standard of care consists of a strict avoidance diet and treatment of severe allergic reactions with epinephrine (adrenaline) or, for milder symptoms, antihistamines.

Although a large number of foods have been reported to cause allergic reactions, cow's milk, chicken's egg, and peanuts are the 3 most common food allergens in the United States. A few foods constitute 90% of food allergies in children: peanut, tree nut, milk (such as cow's milk), egg (such as chicken's egg), soy, wheat, and shellfish (such as shrimp). Tree nuts include, among other nuts, almond, cashew, pecan, pistachio, walnut, and hazelnut. Other common food allergies include sesame seeds and fish, such as codfish and salmon. While some of these food allergies, including milk (for example cow's milk), egg (for example chicken's egg), wheat, and soy, tend to resolve with age, others, such as peanut, tree nut, and shellfish, tend to persist into adulthood.

### III. SUBLINGUAL ADMINISTRATION OF FOOD ALLERGEN

Food allergens for use in treating a food allergy, including peanut allergy, in a human patient in need thereof using sublingual delivery are provided. In some embodiments, a human subject is given very small amounts of a food allergen, such as peanut extract under his or her tongue, and asked to hold the extract there for a certain amount of time (ideally 2 minutes) before swallowing it. With administration to children, it can be difficult to determine exactly how long the extract is held under the tongue, which it is believed will not have a material impact on administration. In comparison with an oral immunotherapy (OIT) approach, sub-lingual immunotherapy (SLIT) has an increased convenience, a decreased risk for systemic reactions, and possibly, a primary effect on mucosal immune responses. It should be understood that while the following description is focused on treatment of peanut allergy with sublingual immunotherapy (to serve as an exemplification of the embodiments only), the food allergens for use in methods of treatment of food allergy disclosed herein are not limited to peanut allergy and apply equally to other food allergies, including, for example, almond, cashew, egg (including chicken's egg), milk (including cow's milk), tree nuts (including hazelnut, pecan, pistachio, and walnut), sesame seed, fish (including salmon, codfish), shellfish (including shrimp), soy, and wheat.

### A. Allergen (for Example Peanut) Protein

Food allergen may refer to the food itself in a minimally processed preparation, such as ground food like peanut flour (which may be in powder form or suspended in a liquid), one or more proteins from the food in a mixture, or a single protein from the food. To be clear, an “allergen protein” such as “peanut protein” as used herein refers, in some embodiments, to one food allergen protein, and in other embodiments, to two or more food allergen proteins. Allergen in the form of a minimally processed preparation of one or more proteins may be provided for treatment of food allergies. Allergen proteins may be in the form of an extract derived from the food. In some embodiments, the allergen protein may be purified and in other embodiments the protein may be a crude extract from the food. In some embodiments in which the protein is a crude extract from the food, the protein may be mixed with non-proteins. The allergen protein may be in the form of a liquid, including the form of sublingual drops.

In some embodiments of the food allergens for use in the treatment of food allergy provided herein, allergen protein (such as peanut protein) is an extract derived from the food (such as peanuts). In some embodiments, the allergen protein (such as peanut protein) is a purified allergen protein. In some embodiments, the allergen protein (such as peanut protein) comprises crude allergen extract. In some embodiments, the allergen protein (such as peanut protein) is the form of allergen (such as peanut) extract liquid. In some embodiments the allergen (such as peanut protein) is in tablet form. In some embodiments, the allergen (such as peanut) extract liquid is in the form of sublingual drops. In some embodiments, sublingual drops may be obtained from Greer Laboratories, Inc (Lenoir, NC). In some embodiments, the bulk material arrives as a 5000 mcg/ml stock solution of allergen (such as peanut) extract, in a 50 ml sterile glass bottles. For more information of the manufacturing of drug substance please refer to the BLA STN BL-101833/1126.0 held by Greer Laboratories. Presence of Ara h1 protein is used as a confirmation of peanut extract.

In some embodiments involving food allergen, sublingual drops comprise crude food allergen (such as peanut) extract (1:20 w/v) fully dissolved in 0.2% phenol and 50%-55% glycerinated saline to a maximum allergen (such as peanut) protein concentration of 5000 mcg/ml. In some embodiments, the Ara h 2 content is estimated to be 6% of allergen (such as peanut) protein concentration. In some embodiments, sublingual liquid drops are taken in the form of pumps. In some embodiments, dilutions are made using glycerinated saline and doses were prescribed as 1 to 8 pumps (50 microliters/pump) of a 1/1000, 1/100, 1/10, or stock dilution of allergen (such as peanut) SLIT or placebo. In some embodiments, the drug product consists of 14 different doses of allergen (such as peanut) extract. Each dosage of drug product is supplied in 32.6 mL plastic vials with the pump. Vials and pumps are received from Greer laboratories, Lenoir, NC. The drug product may be supplied as plastic vials filled allergen (such as peanut) extract liquid and capped with the delivery pump system and which should be stored in a cool, dry place at 2-8° C.

In some embodiments, the peanut protein is combined with vitamin D. Any of the food allergy compositions disclosed herein can contain vitamin D as formulated for administration to the patient. The treatments described herein may benefit from the addition of Vitamin D. While not being bound by theory, we believe that Vitamin D will function in these treatments as an immunomodulator, the deficiency of which not only interferes with its general tolerogenic effects but also may increase an individual's susceptibility to gastrointestinal infection, thereby compromising the integrity of the gastrointestinal epithelial barrier and undermining tolerogenic encounters of immune cells with food protein antigens.

### B. Administration as a Liquid

In some embodiments, the liquid allergen protein (such as peanut protein) of the uses described herein are administered from a pump. In some embodiments, pump is primed before use. In some embodiments, priming the pump involves holding the vial in either hand with index finger resting on top of the pump head. In some embodiments, the vial is held over the sink and the head of the pump is swiftly depressed causing drops to come out.

In some embodiments, the liquid allergen protein (such as peanut protein) described herein is administered by opening the mouth and pointing the nozzle behind the lower teeth and under the tongue. In some embodiments, while lifting the tongue, the pump head is depressed a pre-specified number of times, and drops of the allergen protein (such as peanut protein) are dispensed under the tongue.

### C. Other Forms of Allergen Protein

In some embodiments of the food allergen for use in the treatment of food allergy using sublingual delivery provided herein, the allergen protein (such as peanut protein) is administered in dosage forms other than a liquid. In some embodiments, the allergen protein (such as peanut protein) is administered as a tablet. In some embodiments, the allergen protein (such as peanut protein) is administered as a dissolving strip. In some embodiments, the allergen protein (such as peanut protein) is administered as a gum. In some embodiments, the allergen protein (such as peanut protein) is administered as a candy. In some embodiments, the allergen protein (such as peanut protein) is administered as a paste.

### D. Time Under the Tongue

The allergen protein (such as peanut protein) must be placed under the tongue for it to be administered sublingually. Conventionally, in sublingual immunotherapy, the antigen is placed under the tongue for 2 to 3 minutes and then swallowed.

In some embodiments of the food allergen for use in treating peanut allergy provided herein, the allergen protein (such as peanut protein) is held under the tongue for 0 to 4 minutes. In some embodiments, the allergen protein (such as peanut protein) is held under the tongue for up to 3 minutes. In some embodiments, the allergen protein (such as peanut protein) must be held under the tongue for 3 minutes. In some embodiments, the allergen protein (such as peanut protein) is held under the tongue for 2 to 3 minutes. In some embodiments, the allergen protein (such as peanut protein) is held under the tongue for less than 2 minutes. In some embodiments, the allergen protein (such as peanut protein) is held under the tongue for less one 1 minute. In some embodiments, the allergen protein (such as peanut protein) is held under the tongue for 30 seconds or less.

In some embodiments, the allergen protein (such as peanut protein) is held under the tongue and then swallowed. In some embodiments, the sublingual administration comprises holding the allergen protein (such as peanut protein) under the tongue for less than 2 minutes before it is swallowed by the patient. In some embodiments, the sublingual administration comprises holding the allergen protein (such as peanut protein) under the tongue for 30 seconds or less before it is swallowed. In some embodiments, the sublingual administration comprises holding the allergen protein (such as peanut protein) under the tongue for 30 seconds before it is swallowed.

In some embodiments, the allergen protein (such as peanut protein) is held under the tongue in order for the allergen to be delivered sublingually. Once the allergen has been delivered to the patient, any remnants of the allergen protein are then spat out. In some embodiments, the subject cannot eat, drink, or brush their teeth for 15 minutes after the dose is administered.

### E. Dosing Frequency and Duration

The frequency and duration at which the allergen protein (such as peanut protein) is dosed in the present invention may vary depending on the needs of the human subject. In some embodiments, the allergen protein (such as peanut protein) used in the present invention are administered daily, twice a day, every other day, every third day, every weekday, or 1, 2, 3, 4, 5, 6, or 7 times per week.

Throughout this application, we refer to day 1 of week 1 and also week 1. When we refer to week 1 generically (without specifying day 1), we are referring to at least some or all of days 1, 2 or 3 to 7 of week 1. All other references to a week include any or all of the days 1-7 of that week.

In some embodiments, the patient is administered the maintenance dose for at least 20 weeks to at least 26 weeks. In some embodiments, administration of the maintenance dose continues for at least 26 weeks to at least 52 weeks. In some embodiments, the patient is administered the maintenance dose for a minimum of 1 year. In some embodiments, the patient is administered the maintenance dose for a minimum of 3 years. In some embodiments, the patient is administered the maintenance dose for 5 years.

In some embodiments, each dose is administered in one dose. In some embodiments, at least one dose in the therapy is administered in multiple divided doses (i.e., fractionated dosing). In some embodiments, each dose is administered in multiple divided doses. In some embodiments, the multiple divided doses comprise 2, 3, 4, or 5 divided doses that add up to one total dose.

F. Combination with Oral Immunotherapy

In some embodiments, the present invention comprises an oral immunotherapy component. Oral immunotherapy (OIT) has thus far shown significant outcomes in terms of clinical desensitization to certain food allergens. In some embodiments of the food allergen for use in treating a food allergy disclosed herein, the use comprises an initial dose escalation phase in which a patient is treated with oral immunotherapy (OIT). In some embodiments the escalation phase may include treating the patient with sub-lingual immunotherapy (SLIT). In some embodiments, the escalation phase may include a combination of OIT and SLIT. In some embodiments of the food allergen for use in treating a food allergy disclosed herein, the use comprises a maintenance phase in which a patient is treated with oral immunotherapy (OIT) or epicutaneous immunotherapy (EPIT) or combinations thereof. In some embodiments of the food allergen for use in treating a food allergy disclosed herein, the use comprises an initial dose escalation phase and a maintenance phase in which a patient is treated with oral immunotherapy (OIT). In some embodiments, the use comprises a maintenance phase in which a patient is treated with a combination of sublingual immunotherapy (SLIT) and oral immunotherapy (OIT). In some embodiments, the OIT portion of the maintenance phase is subsequent to the SLIT portion of the maintenance phase. In some embodiments, the OIT portion of the maintenance phase precedes the SLIT portion of the maintenance phase.

In some embodiments, the maintenance phase comprises administering sublingually to the patient a daily dose of allergen protein of about 4.0 to about 5.0 mg for 10 to 14 weeks (or for 20 to 26 weeks) and then administering orally to the patient a daily dose of allergen protein of about 250 to about 300 mg for 10 to 14 weeks (or for 20 to 26 weeks). In some embodiments, the maintenance phase comprises administering sublingually to the patient a daily dose of allergen protein of about 5.0 to about 6.0 mg for 10 to 14 weeks (or for 20 to 26 weeks) and then administering orally to the patient a daily dose of allergen protein of about 300 to about 350 mg for 10 to 14 weeks (or for 20 to 26 weeks).

In some embodiments, the maintenance phase comprises administering sublingually to the patient a daily dose of peanut protein of about 4.0 to about 5.0 mg for 10 to 14 weeks (or for 20 to 26 weeks) and then administering orally to the patient a daily dose of peanut protein of about 250 to about 300 mg for 10 to 14 weeks (or for 20 to 26 weeks). In some embodiments, the maintenance phase comprises administering sublingually to the patient a daily dose of peanut protein of about 5.0 to about 6.0 mg for 10 to 14 weeks (or for 20 to 26 weeks) and then administering orally to the patient a daily dose of peanut protein of about 300 to about 350 mg for 10 to 14 weeks (or for 20 to 26 weeks).

### G. Methods of Administration

Provided herein is a food allergen for use in treating peanut allergy in a human patient in need thereof using sublingual delivery of the food allergen. In some embodiments, the food allergen is administered in phases, such as, for example, a first dose phase, a second dose phase, a third dose phase, and a fourth dose phase. In such cases, the phases are administered in order with the second dose phase following the first dose phase, the third dose phase following the second dose phase, and the fourth dose phase following the third dose phase.

Sublingual immunotherapy (SLIT) requires application of an allergen extract in the sublingual space. There are various advantages associated with the sublingual route of administration. The sublingual space is an immunologically privileged site with high concentrations of dendritic cells. These cells require less antigen to obtain systemic protection such that lower dosages of immunogen may be delivered sublingually compared to orally to obtain the same immune response. These include minimal side effects that are typically limited to oropharyngeal itching or tingling. The side effects of sub-lingual administration or typically less than the oral route and the sub-lingual route is generally well tolerated.

### H. Accelerated Up-Dosing

The sublingual delivery of food allergen used in the present invention provides the advantage of an accelerated up-dosing time to maintenance dose, which is then used to continue treating the patient for some length of time (e.g., for about 26 or 52 weeks or longer). The shortened time to a maintenance dose relative to prior treatment protocols allows patients to begin to reap the benefits of the food (such as peanut) allergy treatment faster and provides some protection from food (such as peanut) allergen sooner. Rather than needing, for example, a conventional 26 weeks or longer to reach the maintenance dose, the patient can get to the maintenance dose by, for example, day 26 of up-dosing. If the patient comes into contact with food (such as peanut) allergen inadvertently before the course of treatment is complete, the patient may nevertheless have a reduced allergic reaction and lower risk of anaphylaxis, as a result of the accelerated up-dosing. This faster approach is believed to achieve a functional amount of tolerance sooner so as to provide significantly more safety in the community against an accidental exposure. This faster approach allows as well a better patient adherence to the treatment schedule.

Accelerated up-dosing refers to an up-dosing phase of the present invention to treat food allergy disclosed herein that is significantly shorter, i.e., 60 days, optionally, 45 days or further optionally 22 to 30 days, preferably 24 to 28 days, compared to conventional up-dosing in immunotherapy. During the accelerated up-dosing phase, progressively greater amounts of a food allergen protein corresponding to the food allergy to be treated is administered sublingually in order to desensitize the patient to the food allergen in a progressive manner. The food allergen is administered according to an accelerated up-dosing phase in which an allergen protein is administered sublingually. In some embodiments, the treatment protocol comprises an accelerated up-dosing phase in which an allergen protein is administered sublingually, and a further maintenance phase. In a further embodiment, there is provided an initial dose escalation phase prior to the accelerated up-dosing and, further optionally, a maintenance phase. In this embodiment, the initial dose escalation phase may be over 1 day or, preferably, over 2 days. The dose escalation phase may comprise an initial dose on day 1 of 0.1 mg followed by a dose of 0.3 mg on day 2. In some embodiments, the present invention provides an accelerated up-dosing phase of between 26 to 28 days. Preferably the up-dosing phase is only 26 days. In some embodiments, some patients may require an extension to the up-dosing phase of up to a further one or two weeks in order to achieve a higher maintenance dose.

In some embodiments, the invention comprises at least these three phases: (a) an initial dose escalation phase, (b) up-dosing phase in which an allergen protein is administered sublingually (also referred to as the accelerated up-dosing), and (c) a maintenance phase. In some embodiments, the (a) initial dose escalation phase is immediately followed by (b) up-dosing phase, which is immediately followed by (c) the maintenance phase.

In some embodiments, the allergen protein is administered orally, subcutaneously, sublingually, or epicutaneously (i.e., via OIT, subcutaneous immunotherapy (SCIT), SLIT, or EPIT, respectively) during the initial dose escalation phase and/or the maintenance phase. In some embodiments, the allergen protein is administered sublingually in the initial dose escalation phase and maintenance phase (i.e., the allergen protein is administered sublingually in all three of the at least three phases of the invention). In some embodiments, the patient is administered a dose of allergen protein daily in the at least three phases of the treatment protocol.

In some embodiments, at least one dose of allergen protein administered to the patient during the up-dosing phase is higher than any dose administered during an initial dose escalation phase, but lower than a dose administered during the maintenance phase. In some embodiments, multiple doses of allergen protein are administered to the patient during the up-dosing phase (also referred to as the accelerated up-dosing). In some embodiments, every dose administered in the up-dosing phase is higher than the immediately preceding dose. In some embodiments, the maintenance dose is the same as the highest dose in the up-dosing phase. In some embodiments, the maintenance dose is higher than the highest dose in the up-dosing phase. In some embodiments, the dose administered during the maintenance phase remains constant throughout the maintenance phase.

In some embodiments, the present invention comprises an up-dosing phase of about 60 days, optionally 45 days, or further optionally 24 to 28 days in which an allergen protein is administered sublingually. In some embodiments, the food allergy is a peanut allergy. In some embodiments, the food allergen is peanut protein. Optionally, the present invention further comprises an initial dose escalation phase of 1 or 2 days before the start of the up-dosing phase, and a maintenance phase of at least about 26 or 52 weeks following the up-dosing phase.

In some embodiments, the initial dose escalation phase comprises administering sublingually to the patient a first dose of allergen protein of about 0.1 mg and a second dose of allergen protein of about 0.3 mg, wherein the first and the second dose are administered both on day 1 or the first dose is administered on day 1 and the second dose is administered on day 2. In some embodiments the up-dosing phase comprises administering sublingually to the patient at least the following four different doses of allergen protein: (a) a first dose phase comprising a first dose of 0.4 to 0.9 mg given daily for 5 to 7 days, (b) a second dose phase comprising a second dose of 1.0 to 2.0 mg given daily for 5 to 7 days, (c) a third dose phase comprising a third dose of 2.1 to 3.4 mg given daily for 5 to 7 days, and (d) a fourth dose phase comprising a fourth dose of 3.5 mg to 4.5 given daily for 5 to 7 days. In some embodiments the up-dosing phase comprises administering sublingually to the patient at least the following four different doses of allergen protein: (a) a first dose phase comprising a first dose of about 0.5 mg given daily for 5 to 7 days, (b) a second dose phase comprising a second dose of about 1.5 mg given daily for 5 to 7 days, (c) a third dose of about 2.5 mg given daily for 5 to 7 days, and (d) a fourth dose phase comprising a fourth dose of about 4.0 mg given daily for 5 to 7 days. In some embodiments, the maintenance phase comprises administering sublingually to the patient a daily dose of allergen protein of about 4.0 to about 5.0 mg for 20 to 28 weeks, preferably for 26 weeks or for 48 to 56 weeks, preferably 52 weeks. In some embodiments, the maintenance phase comprises administering sublingually to the patient a daily dose of allergen protein of about 4.0 to about 5.0 mg for 10 to 14 weeks (or for 20 to 26 weeks) and then administering orally to the patient a daily dose of allergen protein of about 250 to about 300 mg for 10 to 14 weeks (or for 20 to 26 weeks).

In some embodiments, (i) the initial dose escalation phase comprises administering sublingually to the patient a first dose of allergen protein of 0.1 mg and a second dose of allergen protein of 0.3 mg, wherein the first and the second dose are administered both on day 1 or the first dose is administered on day 1 and the second dose is administered on day 2, (ii) the up-dosing phase comprises administering sublingually to the patient: (a) a first dose phase comprising a first dose of 0.5 mg given daily for 5 to 7 days, (b) a second dose phase comprising a second dose of 1.5 mg given daily for 5 to 7 days, (c) a third dose phase comprising a third dose of 2.5 mg given daily for 5 to 7 days, and (d) a fourth dose phase comprising a fourth dose of 4.0 mg given daily for 5 to 7 days, and (iii) the maintenance phase comprises either (a) administering sublingually to the patient a daily dose of allergen protein of 4.0 or 5.0 mg for about 26 or 52 weeks or (b) administering sublingually to the patient a daily dose of allergen protein of 4.0 or 5.0 mg for 10 to 14 weeks (or for 20 to 26 weeks) and then administering orally to the patient a daily dose of allergen protein of 250 mg for 10 to 14 weeks (or for 20 to 26 weeks).

In some embodiments, the initial dose escalation phase comprises administering sublingually to the patient a first dose of allergen protein of about 0.3 mg and a second dose of allergen protein of about 0.9 mg, wherein the first and the second dose are administered both on day 1 or the first dose is administered on day 1 and the second dose is administered on day 2. In some embodiments, the up-dosing phase comprises administering sublingually to the patient at least the following four different doses of allergen protein: (a) a first dose phase comprising a first dose of 1.0 to 1.5 mg given daily for 5 to 7 days, (b) a second dose phase comprising a second dose of 1.6 to 2.9 mg given daily for 5 to 7 days, (c) a third dose phase comprising a third dose of 3.0 to 4.0 mg given daily for 5 to 7 days, and (d) a fourth dose phase comprising a fourth dose of 4.1 to 6.0 mg given daily for 5 to 7 days. In some embodiments, the up-dosing phase comprises administering sublingually to the patient at least the following four different doses of allergen protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days In some embodiments, the maintenance phase comprises administering sublingually to the patient a daily dose of allergen protein of about 5.0 to about 6.0 mg for about 26 or 52 weeks. In some embodiments, the maintenance phase comprises administering sublingually to the patient a daily dose of allergen protein of about 5.0 to about 6.0 mg for 10 to 14 weeks (or for 20 to 26 weeks) and then administering orally to the patient a daily dose of allergen protein of about 300 to about 350 mg for 10 to 14 weeks (or for 20 to 26 weeks).

In some embodiments, (i) the initial dose escalation phase comprises administering sublingually to the patient a first dose of allergen protein of 0.3 mg and a second dose of allergen protein of 0.9 mg, wherein the first and the second dose are administered both on day 1 or the first dose is administered on day 1 and the second dose is administered on day 2, (ii) the up-dosing phase comprises administering sublingually to the patient at least the following four different doses of allergen protein: (a) a first dose phase comprising a first dose of 1.2 mg given daily for 5 to 7 days, (b) a second dose phase comprising a second dose of 2.5 mg given daily for 5 to 7 days, (c) a third dose phase comprising a third dose of 3.5 mg given daily for 5 to 7 days, and (d) a fourth dose phase comprising a fourth dose of 5.0 mg given daily for 5 to 7 days, and (iii) the maintenance phase comprises either (a) administering sublingually to the patient a daily dose of allergen protein of 5.0 or 6.0 mg for about 26 or 52 weeks or (b) administering sublingually to the patient a daily dose of allergen protein of 5.0 to 6.0 mg for 10 to 14 weeks (or for 20 to 26 weeks) and then administering orally to the patient a daily dose of allergen protein of 300 mg for 10 to 14 weeks (or for 20 to 26 weeks).

In some embodiments, the initial dose escalation phase comprises administering sublingually to the patient a first dose of peanut protein of about 0.1 mg and a second dose of peanut protein of about 0.3 mg, wherein the first and the second dose are administered both on day 1 or the first dose is administered on day 1 and the second dose is administered on day 2. In some embodiments the up-dosing phase comprises administering sublingually to the patient at least the following four different doses of peanut protein: (a) a first dose phase comprising a first dose of 0.4 to 0.9 mg given daily for 5 to 7 days, (b) a second dose phase comprising a second dose of 1.0 to 2.0 mg given daily for 5 to 7 days, (c) a third dose phase comprising a third dose of 2.1 to 3.4 mg given daily for 5 to 7 days, and (d) a fourth dose phase comprising a fourth dose of 3.5 mg to 4.5 given daily for 5 to 7 days. In some embodiments the up-dosing phase comprises administering sublingually to the patient at least the following four different doses of peanut protein: (a) a first dose phase comprising a first dose of about 0.5 mg given daily for 5 to 7 days, (b) a second dose phase comprising a second dose of about 1.5 mg given daily for 5 to 7 days, (c) a third dose of about 2.5 mg given daily for 5 to 7 days, and (d) a fourth dose phase comprising a fourth dose of about 4.0 mg given daily for 5 to 7 days. In some embodiments, the maintenance phase comprises administering sublingually to the patient a daily dose of peanut protein of about 4.0 to about 5.0 mg for 26 or 52 weeks. In some embodiments, the maintenance phase comprises administering sublingually to the patient a daily dose of peanut protein of about 4.0 to about 5.0 mg for 10 to 14 weeks (or for 20 to 26 weeks) and then administering orally to the patient a daily dose of peanut protein of about 250 to about 300 mg for 10 to 14 weeks (or for 20 to 26 weeks).

In some embodiments, (i) the initial dose escalation phase comprises administering sublingually to the patient a first dose of peanut protein of 0.1 mg and a second dose of peanut protein of 0.3 mg, wherein the first and the second dose are administered both on day 1 or the first dose is administered on day 1 and the second dose is administered on day 2, (ii) the up-dosing phase comprises administering sublingually to the patient: (a) a first dose phase comprising a first dose of 0.5 mg given daily for 5 to 7 days, (b) a second dose phase comprising a second dose of 1.5 mg given daily for 5 to 7 days, (c) a third dose phase comprising a third dose of 2.5 mg given daily for 5 to 7 days, and (d) a fourth dose phase comprising a fourth dose of 4.0 mg given daily for 5 to 7 days, and (iii) the maintenance phase comprises either (a) administering sublingually to the patient a daily dose of peanut protein of 4.0 or 5.0 mg for about 26 or 52 weeks or (b) administering sublingually to the patient a daily dose of peanut protein of 4.0 or 5.0 mg for 10 to 14 weeks (or for 20 to 26 weeks) and then administering orally to the patient a daily dose of peanut protein of 250 mg for 10 to 14 weeks (or for 20 to 26 weeks).

In some embodiments, the initial dose escalation phase comprises administering sublingually to the patient a first dose of peanut protein of about 0.3 mg and a second dose of peanut protein of about 0.9 mg, wherein the first and the second dose are administered both on day 1 or the first dose is administered on day 1 and the second dose is administered on day 2. In some embodiments, the up-dosing phase comprises administering sublingually to the patient at least the following four different doses of peanut protein: (a) a first dose phase comprising a first dose of 1.0 to 1.5 mg given daily for 5 to 7 days, (b) a second dose phase comprising a second dose of 1.6 to 2.9 mg given daily for 5 to 7 days, (c) a third dose phase comprising a third dose of 3.0 to 4.0 mg given daily for 5 to 7 days, and (d) a fourth dose phase comprising a fourth dose of 4.1 to 6.0 mg given daily for 5 to 7 days. In some embodiments, the up-dosing phase comprises administering sublingually to the patient at least the following four different doses of peanut protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days. In some embodiments, the maintenance phase comprises administering sublingually to the patient a daily dose of peanut protein of about 5.0 to about 6.0 mg for about 26 or 52 weeks. In some embodiments, the maintenance phase comprises administering sublingually to the patient a daily dose of peanut protein of about 5.0 to about 6.0 mg for 10 to 14 weeks (or for 20 to 26 weeks) and then administering orally to the patient a daily dose of peanut protein of about 300 to about 350 mg for 10 to 14 weeks (or for 20 to 26 weeks).

In some embodiments, (i) the initial dose escalation phase comprises administering sublingually to the patient a first dose of peanut protein of 0.3 mg and a second dose of peanut protein of 0.9 mg, wherein the first and the second dose are administered both on day 1 or the first dose is administered on day 1 and the second dose is administered on day 2, (ii) the up-dosing phase comprises administering sublingually to the patient at least the following four different doses of peanut protein: (a) a first dose phase comprising a first dose of 1.2 mg given daily for 5 to 7 days, (b) a second dose phase comprising a second dose of 2.5 mg given daily for 5 to 7 days, (c) a third dose phase comprising a third dose of 3.5 mg given daily for 5 to 7 days, and (d) a fourth dose phase comprising a fourth dose of 5.0 mg given daily for 5 to 7 days, and (iii) the maintenance phase comprises either (a) administering sublingually to the patient a daily dose of peanut protein of 5.0 or 6.0 mg for about 26 or 52 weeks or (b) administering sublingually to the patient a daily dose of peanut protein of 5.0 to 6.0 mg for 10 to 14 weeks (or for 20 to 26 weeks) and then administering orally to the patient a daily dose of peanut protein of 300 mg for 10 to 14 weeks (or for 20 to 26 weeks).

In some embodiments, the food allergen for use in treating peanut allergy in a human patient are provided wherein: on day 1 of week 1, administering peanut protein at a dose of 5-20 mcg to the patient, on at least some of days 2-7 in week 1, administering peanut protein at a dose of 50-200 mcg to the patient, in week 2, administering peanut protein at a dose of 400-600 mcg to the patient, in week 3, administering peanut protein at a dose of 1500-3000 mcg to the patient, in week 4, administering peanut protein at a dose of 3500-6000 mcg to the patient, wherein the peanut protein is administered sublingually. In some embodiments, the dose administered in week 4 is the maintenance dose. In some embodiments, a maintenance dose is higher than the dose administered in week 4. In some embodiments, a maintenance dose is lower than the dose administered in week 4. In some embodiments, at least one dose increase occurs within at least one week. In some embodiments, at least one dose increase occurs within each of weeks 1, 2, and 3. In some embodiments, the dose remains the same within at least one week. In some embodiments, the dose remains the same within each of weeks 1, 2, and 3, respectively.

In some embodiments, the food allergen for use in treating peanut allergy in a human patient comprises: on day 1 of week 1, administering peanut protein at a dose of 10 mcg to the patient, on at least some of days 2-7 in week 1, administering peanut protein at a dose of 100 mcg to the patient, in week 2, administering peanut protein at a dose of 500 mcg to the patient, in week 3, administering peanut protein at a dose of 2500 mcg to the patient, in week 4, administering peanut protein at a dose of 4000 mcg to the patient.

In some embodiments, the present invention comprises: on day 1 of week 1, administering peanut protein at a dose of 10 mcg to the patient, on days 2-7 of week 1, administering peanut protein at a dose of 100 mcg to the patient, on days 1-7 of week 2, administering peanut protein at a dose of 500 mcg to the patient, on days 1-7 of week 3, administering peanut protein at a dose of 2500 mcg to the patient, on days 1-7 of week 4, administering peanut protein at a dose of 4000 mcg to the patient, wherein the peanut protein is administered as a liquid sublingually daily, each dose is administered in one dose, the dose remains the same within each of weeks 1, 2, and 3, respectively, the dose administered in week 4 is the maintenance dose, administration of the maintenance dose continues for 1 year.

### I. Elevated Starting Dose

In some embodiments the sublingual delivery of food antigen provides the advantage of an elevated starting dose, such that the patient also receives the benefit of an accelerated time to maintenance dose, which is then used to continue treating the patient for some length of time (e.g., 26 or 52 weeks). These are based on the premise that patients do not generally need to start at such a low dose due to the relative safety of a sublingual route of administration. The higher starting dose and/or the shortened time to a maintenance dose relative to prior therapies allows patients to begin to reap the benefits of the peanut allergy treatment even faster and provides some protection from peanut allergen sooner. From early in the treatment protocol (e.g., day 1 or days 2-7 of week 1), the patient can begin to be desensitized to peanut protein more aggressively compared to prior treatment protocols. Further, due to the higher starting dose, rather than needing, for example, 26 weeks to reach the maintenance dose, the patient can get to the maintenance dose by, for example, week 4. If the patient comes into contact with peanut allergen inadvertently before the course of treatment is complete, the patient may nevertheless have a reduced allergic reaction and lower risk of anaphylaxis.

In some embodiments, the food allergen for use in treating food allergy in a human patient are provided wherein the first dose of allergen protein (such as peanut protein) is about 0.1 mg and a second dose of allergen protein of about 0.3 mg, wherein the first and the second dose are administered both on day 1 or the first dose is administered on day 1 and the second dose is administered on day 2. In other embodiments, the first dose of allergen protein is about 0.3 mg and the second dose of allergen protein of about 0.9 mg, wherein the first and the second dose are administered both on day 1 or the first dose is administered on day 1 and the second dose is administered on day 2.

In some embodiments, the food allergen for use in treating peanut allergy in a human patient are provided wherein: on day 1 of week 1, administering peanut protein at a dose of 500-2000 mcg to the patient, on at least some of days 2-7 in week 1, administering peanut protein at a dose of 1000-4000 mcg to the patient, in week 2, administering peanut protein at a dose of 1500-5000 mcg to the patient, in week 3, administering peanut protein at a dose of 2000-6000 mcg to the patient, in week 4, administering peanut protein at a dose of 3000-7000 mcg to the patient. In some embodiments, the dose administered in week 4 is the maintenance dose. In some embodiments, a maintenance dose is higher than the dose administered in week 4. In some embodiments, a maintenance dose is lower than the dose administered in week 4. In some embodiments, the protocol comprises: on day 1 of week 1, administering peanut protein at a dose of 1000 mcg to the patient, on at least some of days 2-7 in week 1, administering peanut protein at a dose of 2000 mcg to the patient, in week 2, administering peanut protein at a dose of 2500 mcg to the patient, in week 3, administering peanut protein at a dose of 3500 mcg to the patient, in week 4, administering peanut protein at a dose of 4000 mcg to the patient.

In some embodiments, the food allergen for use in treating peanut allergy in a human patient are provided comprising: on day 1 of week 1, administering peanut protein at a dose of 1000 mcg to the patient, on days 2-7 of week 1, administering peanut protein at a dose of 2000 mcg to the patient, on days 1-7 of week 2, administering peanut protein at a dose of 2500 mcg to the patient, on days 1-7 of week 3, administering peanut protein at a dose of 3500 mcg to the patient, on days 1-7 of week 4, administering peanut protein at a dose of 4000 mcg to the patient, wherein the peanut protein is administered as a liquid sublingually daily, each dose is administered in one dose, the dose remains the same within each of weeks 1, 2, and 3, respectively, the dose administered in week 4 is the maintenance dose, administration of the maintenance dose continues for 26 or 52 weeks.

### J. Based on Oral Food Challenge

In some aspects of the present invention, the sublingual delivery of food antigen provides the advantage of dividing the patient population into groups who undergo different treatment protocols depending on the degree of severity of their peanut allergy. This approach is based on the premise that a one-size-fits-all approach both misses the opportunity to allow less severely allergic patients to reach a maintenance dose and reap benefits of the treatment faster, while also subjecting more severely allergic patients to a riskier treatment that could trigger allergic reactions, including anaphylaxis. These approaches contemplate tracking patients, wherein each patient can be put on a sublingual treatment that best suits their level of peanut allergy. In some embodiments of the present invention, an oral food challenge (OFC) is administered before a treatment protocol is selected and the patient's performance on the OFC is used as a proxy for the patient's level of allergy. The OFC is used to evaluate the patient's sensitivity to peanut antigens and the treatment protocol is selected based on the dose at which the patient “fails” the OFC, i.e. based on the ED.

In some embodiments of the present invention (a) the patient is administered an initial oral food challenge prior to treatment and (b) the treatment protocol varies depending on the dose at which the patient reaches an eliciting dose (ED) in the oral food challenge. In some embodiments, the initial oral food challenge administered prior to the treatment is administered in order to evaluate the patient's sensitivity to peanut antigens by determining the patient's eliciting dose (ED), and the ED is used to select an appropriate treatment protocol for the patient. In some embodiments of the present invention, the use comprises evaluating the patient's sensitivity to an antigen comprising administering an initial oral food challenge so as to determine the eliciting dose (ED) for the patient, and the selection of doses within the treatment protocol depends on the patient's eliciting dose (ED).

In some embodiments of the present invention, the first dose administered to the patient in the initial dose escalation phase is about 0.1% of the patient's ED. In some embodiments, the first dose in the escalation phase is 0.1% of the ED. For instance, in some embodiments, if the ED is 100 mg the first dose is of 0.1 mg, whereas if the ED is 300 mg the first dose is of 0.3 mg.

In some embodiments, if the patient has a relatively lower eliciting dose (ED), such as ≤100 mg, the treatment protocol comprises any one or more of the following phases: (i) an initial dose escalation phase comprising administering sublingually to the patient a first dose of allergen protein of about 0.1 mg and a second dose of allergen protein of about 0.3 mg, wherein the first and the second dose are administered both on day 1 or the first dose is administered on day 1 and the second dose is administered on day 2, (ii) an up-dosing phase comprising (a) a first dose phase comprising a first dose of about 0.5 mg given daily for 5 to 7 days, (b) a second dose phase comprising a second dose of about 1.5 mg given daily for 5 to 7 days, (c) a third dose phase comprising a third dose of about 2.5 mg given daily for 5 to 7 days, and (d) a fourth dose phase comprising a fourth dose of about 4.0 mg given daily for 5 to 7 days, and (iii) a maintenance phase comprising administering sublingually to the patient a daily dose of allergen protein of about 4.0 mg to about 5.0 mg for about 26 or 52 weeks

In some embodiments, if the patient has a relatively lower eliciting dose (ED), the present invention comprises (i) an initial dose escalation phase comprising administering sublingually to the patient a first dose of allergen protein of about 0.1 mg and a second dose of allergen protein of about 0.3 mg, wherein the first and the second dose are administered both on day 1 or the first dose is administered on day 1 and the second dose is administered on day 2, (ii) an up-dosing phase comprising (a) a first dose phase comprising a first dose of about 0.5 mg given daily for 5 to 7 days, (b) a second dose phase comprising a second dose of about 1.5 mg given daily for 5 to 7 days, (c) a third dose phase comprising a third dose of about 2.5 mg given daily for 5 to 7 days, and (d) a fourth dose phase comprising a fourth dose of about 4.0 mg given daily for 5 to 7 days, and (iii) a maintenance phase comprising administering sublingually to the patient a daily dose of allergen protein of about 4.0 mg to about 5.0 mg for about 26 or 52 weeks.

If some embodiments, if the patient has a relatively higher eliciting dose (ED), such as 101 mg-300 mg, the present invention comprises any one or more of the following phases: (i) an initial dose escalation phase comprising administering sublingually to the patient a first dose of allergen protein of about 0.3 mg and a second dose of allergen protein of about 0.9 mg, wherein the first and the second dose are administered both on day 1 or the first dose is administered on day 1 and the second dose is administered on day 2, (ii) an up-dosing phase comprises administering sublingually to the patient at least the following four different doses of allergen protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days, and (iii) a maintenance phase comprises administering sublingually to the patient a daily dose of allergen protein of about 5.0 to about 6.0 mg for about 26 or 52 weeks.

If some embodiments, if the patient has a higher eliciting dose (ED), the present invention comprises (i) an initial dose escalation phase comprising administering sublingually to the patient a first dose of allergen protein of about 0.3 mg and a second dose of allergen protein of about 0.9 mg, wherein the first and the second dose are administered both on day 1 or the first dose is administered on day 1 and the second dose is administered on day 2, (ii) an up-dosing phase comprises administering sublingually to the patient at least the following four different doses of allergen protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days, and (iii) a maintenance phase comprises administering sublingually to the patient a daily dose of allergen protein of about 5.0 mg to about 6.0 mg for about 26 or 52 weeks.

In some embodiments, if the patient has a relatively lower eliciting dose (ED), such as ≤100 mg, the present invention comprises any one or more of the following phases: (i) an initial dose escalation phase comprising administering sublingually to the patient a first dose of peanut protein of about 0.1 mg and a second dose of peanut protein of about 0.3 mg, wherein the first and the second dose are administered both on day 1 or the first dose is administered on day 1 and the second dose is administered on day 2, (ii) an up-dosing phase comprising (a) a first dose phase comprising a first dose of about 0.5 mg given daily for 5 to 7 days, (b) a second dose phase comprising a second dose of about 1.5 mg given daily for 5 to 7 days, (c) a third dose phase comprising a third dose of about 2.5 mg given daily for 5 to 7 days, and (d) a fourth dose phase comprising a fourth dose of about 4.0 mg given daily for 5 to 7 days, and (iii) a maintenance phase comprising administering sublingually to the patient a daily dose of peanut protein of about 4.0 mg to about 5.0 for about 26 or 52 weeks.

In some embodiments, if the patient has a relatively lower eliciting dose (ED), the invention comprises (1) an initial dose escalation phase comprising administering sublingually to the patient a first dose of peanut protein of about 0.1 mg and a second dose of peanut protein of about 0.3 mg, wherein the first and the second dose are administered both on day 1 or the first dose is administered on day 1 and the second dose is administered on day 2, (ii) an up-dosing phase comprising (a) a first dose phase comprising a first dose of about 0.5 mg given daily for 5 to 7 days, (b) a second dose phase comprising a second dose of about 1.5 mg given daily for 5 to 7 days, (c) a third dose phase comprising a third dose of about 2.5 mg given daily for 5 to 7 days, and (d) a fourth dose phase comprising a fourth dose of about 4.0 mg given daily for 5 to 7 days, and (iii) a maintenance phase comprising administering sublingually to the patient a daily dose of peanut protein of about 4.0 mg to about 5.0 mg for about 26 or 52 weeks.

In some embodiments, if the patient has a relatively higher eliciting dose (ED), such as 101 mg-300 mg, the present invention comprises any one or more of the following phases: (i) an initial dose escalation phase comprising administering sublingually to the patient a first dose of peanut protein of about 0.3 mg and a second dose of peanut protein of about 0.9 mg, wherein the first and the second dose are administered both on day 1 or the first dose is administered on day 1 and the second dose is administered on day 2, (ii) an up-dosing phase comprises administering sublingually to the patient at least the following four different doses of peanut protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days, and (iii) a maintenance phase comprises administering sublingually to the patient a daily dose of peanut protein of about 5.0 mg to about 6.0 mg for about 26 or 52 weeks.

In some embodiments, if the patient has a higher eliciting dose (ED), the present invention comprises (i) an initial dose escalation phase comprising administering sublingually to the patient a first dose of peanut protein of about 0.3 mg and a second dose of peanut protein of about 0.9 mg, wherein the first and the second dose are administered both on day 1 or the first dose is administered on day 1 and the second dose is administered on day 2, (ii) an up-dosing phase comprises administering sublingually to the patient at least the following four different doses of peanut protein: (a) a first dose phase comprising a first dose of about 1.2 mg given daily for 5 to 7 days, (b) a second dose phase comprising a second dose of about 2.5 mg given daily for 5 to 7 days, (c) a third dose phase comprising a third dose of about 3.5 mg given daily for 5 to 7 days, and (d) a fourth dose phase comprising a fourth dose of about 5.0 mg given daily for 5 to 7 days, and (iii) a maintenance phase comprises administering sublingually to the patient a daily dose of peanut protein of about 5.0 mg to about 6.0 mg for about 26 or 52 weeks.

### K. Prevention of Food (for Example Peanut) Allergy

In some aspects of the present invention, administering food allergen sublingual prevents food (such as peanut) allergy for developing in a human patient who has not previously been diagnosed as having a food allergy. The human patient may be, for example, a child (i.e., 18 years old or less) or an infant (i.e., aged less than 4 years old) at an elevated risk of developing food (such as peanut) allergy. Studies have shown that children who have other allergies or whose parents have food or environmental allergies are at an elevated risk of developing food (such as peanut) allergy. Sublingual dosing may align better with exclusive breastfeeding for infants, including infants at risk of developing allergy, compared to other types of allergy treatment, such as oral immunotherapy, due to ease of administration.

In some aspects of the present invention the food allergen is administered sublingually, wherein the human patient has not previously been diagnosed as having a food allergy. In some embodiments, the human patient is at an elevated risk of developing food allergy relative to the general population. In some embodiments, the food allergen is a liquid. In some embodiments, the patient may be 18 years old or less, preferably 2 to 18 years old, 3 to 18 years old or 4 to 18 years old. In some embodiments, the human patient is an infant 12 months old or younger. In some embodiments, the human patient is an infant from 4 to 6 months old. In some embodiments, the infant is breastfed.

### IV. ORAL FOOD CHALLENGE

An oral food challenge (OFC), or feeding test, is a medical procedure in which a food is eaten slowly, in gradually increasing amounts, under medical supervision, to accurately diagnose or rule out a true food allergy, or to determine an appropriate baseline for treatment of the allergy. An OFC is used as a medical test when the food allergy is in question and will either confirm or rule out the presence of the allergy. An OFC may also be used to determine an appropriate baseline for treatment of the allergy. An OFC may occasionally be administered to patients who are being treated for food allergy (e.g., with SLIT, OIT, SCIT, or EPIT) to determine if the therapy is successful. In some aspects, an initial OFC is administered prior to treatment. In some aspects, a final OFC is administered following treatment. In some aspects, an interim OFC is administering during treatment. In some embodiments, the OFC is double blind placebo controlled, in which case, it can also be referred to as a DBPCFC. In some embodiments, the OFC is unblinded. In some embodiments of the invention of treating herein, the patient failed an oral food challenge prior to treatment. In other embodiments, the patient did not have an oral food challenge prior to treatment. In some embodiments of the invention provided herein, the invention comprises evaluating the subject's sensitivity to peanut antigens comprising administering an initial oral food challenge comprising administering peanut protein. In some embodiments, the subject is said to have failed the OFC when the subject has an allergic reaction to peanut protein. In such cases, the dose at which the subject failed the OFC may be referred to in some cases as the eliciting dose (ED). Specifically, the eliciting dose is the lowest dose at which objective signs/symptoms of an immediate hypersensitivity reaction develop. In some embodiments, the subject stops the OFC before showing any sign of allergic reaction to peanut protein. In some embodiments, the subject stops the OFC at the dose immediately preceding the dose at which the patient has an allergic reaction to peanut protein.

In some embodiments, the peanut protein administered in the oral food challenge comprises peanut flour. In some embodiments, the peanut protein administered in the oral food challenge comprises a liquid, a tablet, a dissolvable strip, a gum, or a paste. In some embodiments, the oral food challenge comprises administering multiple doses of peanut protein. In some embodiments, the oral food challenge comprises administering multiple doses of peanut flour. In some embodiments, the oral food challenge comprises administering a single dose of peanut protein. In some embodiments, the oral food challenge comprises administering a single dose of peanut flour.

In some embodiments, the present invention comprises evaluating the patient's sensitivity to food (such as peanut) antigens comprising administering an initial oral food challenge, wherein the patient will undergo different treatment protocols depending on the patient's eliciting dose (ED). In some embodiments, the present invention comprises administering sublingually a food allergen (such as peanut protein) to the patient which varies depending on the dose at which the patient reaches an eliciting dose (ED). In some embodiments, the first dose administered during the initial dose escalation phase is about 0.1% of the ED. In some embodiments, the first dose administered during the initial dose escalation phase is 0.1% of the ED.

In some embodiments, wherein the oral food challenge comprises administering increasing amounts of a food allergen (such as peanut protein) doses every 30 minutes to a maximum dose of 300 mg according to the following schedule: 1, 3, 10, 30, 100, and 300 mg of food allergen (such as peanut protein). In some embodiments, the present invention comprises administering different doses depending on whether his or her eliciting dose (ED) is ≤100 mg, is 101-300 mg, or higher.

Preferably the patient is particularly allergic to the food allergen with an eliciting dose of 100 mg of allergen or less, preferably 100 mg of peanut protein or less in the case of peanut allergy.

In some embodiments, wherein the oral food challenge comprises administering increasing food allergen (such as peanut protein) doses every 30 minutes to a maximum dose of 300 mg according to the following schedule: 1, 3, 10, 30, 100, and 300 mg of food allergen (such as peanut protein). In some embodiments, the present invention comprises administering a different treatment protocol depending on whether his or her eliciting dose (ED) is ≤100 mg, is 101-300 mg, or is 301-600 mg.

In some embodiments the present invention comprises evaluating the patient's sensitivity to peanut antigens comprising administering an initial oral food challenge comprising administering multiple doses of peanut protein up to a maximum cumulative total of 50 mg-150 mg or a maximum cumulative total of 150 mg-450 mg. These maximum cumulative doses may be appropriate in clinical practice, where a healthcare provider may want or need to avoid a severe allergic reaction at the clinic. The relationship between challenge dose and cumulative amount of food allergen (such as peanut protein) is well known in the art.

In some embodiments the present invention comprises evaluating the patient's sensitivity to peanut antigens comprising administering an initial oral food challenge comprising administering multiple doses of peanut flour about every 5-60 minutes in increasing amounts up to a maximum cumulative total of 4000-6000 mg peanut protein and characterizing the patient as failing the initial oral food challenge if the patient shows signs of peanut allergy. In some embodiments, the multiple increasing doses comprise 9 increasing doses. In some embodiments, the oral food challenge comprises administering the peanut protein at incremental doses of 1-9 mg, 10-20 mg, 21-75 mg, 76-200 mg, 201-400 mg, 401-750 mg, 751-1250 mg, 1251-1550 mg, and 1551-2000 mg. In some embodiments, the oral food challenge comprises administering the peanut protein at incremental doses of 5 mg, 15 mg, 50 mg, 100 mg, 250 mg, 500 mg, 1000 mg, 1500 mg, and 1580 mg. In some embodiments, the oral food challenge comprises administering increasing incremental doses of peanut flour every 20 minutes.

### V. ADDITIONAL SUBJECT CHARACTERISTICS

In some embodiments, the subject is a human being. In some embodiments, the human subject is an adult. In some embodiments, the subject is an adolescent. In some embodiments, the human subject is a child. In some embodiments, the subject is an infant. For the purposes of the present invention, an infant is less than 4 years old, a child is 18 years old or less and an adolescent is from 13 to 18 years old. In some embodiments, the human patient is 11 years old or under. In some embodiments, the patient is an infant 12 months old or younger. In some embodiments, the human patient is an infant from 4 to 6 months old. In some embodiments, the patients is aged from 4 to 18 years old.

In some embodiments of the food allergen used in treating peanut allergy using sublingual delivery provided herein, the human patient has reflux or eosinophilic esophagitis. In some embodiments the food allergen used in treating peanut allergy using sublingual delivery provided herein, the human patient is or was excluded from oral immunotherapy due to having reflux or eosinophilic esophagitis.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The above statement of invention and description refer to food allergens for use in a method of treating food allergy, in the medical use format required for European patent practice. The present invention also covers equivalent claims to methods of treatment that are allowable in jurisdictions outside Europe such as the USA.

### EXAMPLE

The Examples that follow are illustrative of specific embodiments of the disclosure, and various uses thereof. They are set forth for explanatory purposes only and should not be construed as limiting the scope of the invention in any way.

A double-blinded, placebo-controlled study of peanut sublingual immunotherapy followed by a 52-week open label maintenance period is conducted in order to assess safety and tolerability of accelerated up-dosing of SLIT therapy in patients with peanut allergy.

Patients are initiated on 0.1 mg SLIT or placebo on day 1 of treatment. Initial dose escalation begins on day 2 to 0.3 mg. The patients that withstand the rush phase with no anaphylaxis or adverse events then proceed to the 26th day accelerated up-dosing period with dose increases every 1 week until the dosage reaches 4.0 mg (or placebo). On day 29 the patients enter into the maintenance weeks. At the end of 52 weeks of open SLIT study drug administration, the subjects undergo a DBPCFC to assess for levels of sensitization as determined by the eliciting dose according to the protocol.

The treatment protocol is shown in FIG. 1.

Subjects are eligible for the study if they fulfil all of the following inclusion criteria:
1. Age 4 to 18 years, either sex, any race, any ethnicity;
2. Convincing clinical history of peanut allergy or an in vitro peanut-specific IgE>0.35 kUA/L determined by a commercial system within the past 12 months; and
3. A positive result to 100 mg peanut protein DBPCFC after enrollment (during screening visit, i.e. ≤100 mg ED).
4. Written consent from participant's parent/guardian, including assent where indicated.

Levels of sensitization are assessed by measurement of total IgE, peanut specific IgE and IgG4 in samples from the subject. These biomarkers are measured by known methods and assessed against known indicative levels of sensitization. These biomarkers are collected at baseline, end of Part A, 26 weeks, and 52 weeks (end of Part B).

1. A method of treating a peanut allergy in a human patient, wherein the treatment comprises sublingual administration of a food allergen in an up-dosing phase followed by a maintenance phase, wherein the up-dosing phase is of 24 to 28 days, and wherein the food allergen is peanut protein.

2-4. (canceled)

5. The method of claim 1, wherein the treatment further comprises an initial dose escalation phase of 1 or 2 days before the up-doing phase.

6. The method of claim 5, wherein the initial dose escalation phase comprises administering sublingually to the human patient a first dose of peanut protein of about 0.1 mg and a second dose of peanut protein of about 0.3 mg, wherein the first and the second dose are administered both on day 1, or the first dose is administered on day 1 and the second dose is administered on day 2.

7. The method of claim 1, wherein the up-dosing phase comprises administering sublingually to the human patient at least the following four different doses of peanut protein:
a) a first dose phase comprising a first dose of about 0.5 mg administered daily for 5 to 7 days,
b) a second dose phase comprising a second dose of about 1.5 mg administered daily for 5 to 7 days,
c) a third dose phase comprising a third dose of about 2.5 mg administered daily for 5 to 7 days, and
d) a fourth dose phase comprising a fourth dose of about 4.0 mg administered daily for 5 to 7 days.

8. The method of claim 1, wherein the maintenance phase comprises administering sublingually to the human patient a daily dose of peanut protein of about 4.0 mg to about 5.0 mg for at least 20 to 28 weeks.

9. The method of claim 5, wherein the initial dose escalation phase comprises administering sublingually to the human patient a first dose of peanut protein of about 0.3 mg and a second dose of peanut protein of about 0.9 mg, wherein the first and the second dose are administered both on day 1, or the first dose is administered on day 1 and the second dose is administered on day 2.

10. The method of claim 1, wherein the up-dosing phase comprises administering sublingually to the human patient at least the following four different doses of peanut protein:
a) a first dose phase comprising a first dose of about 1.2 mg administered daily for 5 to 7 days,
b) a second dose phase comprising a second dose of about 2.5 mg administered daily for 5 to 7 days,
c) a third dose phase comprising a third dose of about 3.5 mg administered daily for 5 to 7 days, and
d) a fourth dose phase comprising a fourth dose of about 5.0 mg administered daily for 5 to 7 days.

11. The method of claim 1, wherein the maintenance phase comprises administering sublingually to the human patient a daily dose of peanut protein of about 5.0 mg to about 6.0 mg for at least 20 to 28 weeks.

12. The method of claim 5, wherein the first dose administered in the initial dose escalation phase is about 0.1% of the human patient's eliciting dose (ED).

13. The method of claim 12, wherein the human patient's ED is ≤100 mg.

14. The method of claim 12, wherein the human patient's ED is 101 mg to 300 mg.

15. The method of claim 1, wherein the maintenance phase comprises administering sublingually to the human patient a daily dose of peanut protein of about 5.0 mg to about 6.0 mg for 20 to 28 weeks, or for 20 to 28 weeks and for one additional period of 20 to 28 weeks.

16. A method of treating a peanut allergy in a human patient, wherein the treatment comprises sublingual administration of a peanut protein, and the treatment comprises:
a) an initial dose escalation phase that comprises administering sublingually to the patient a first dose of peanut protein of about 0.1 mg on day 1, and a second dose of allergen protein of about 0.3 mg on day 2;
b) an up-dosing phase that comprises administering sublingually to the patient at least the following four different doses of peanut protein:
i. a first dose phase comprising a first dose of about 0.5 mg administered daily for 7 days,
ii. a second dose phase comprising a second dose of about 1.5 mg administered daily for 7 days,
iii. a third dose phase comprising a third dose of about 2.5 mg administered daily for 7 days, and
iv. a fourth dose phase comprising a fourth dose of about 4.0 mg administered daily for 7 days; and
c) a maintenance phase that comprises administering sublingually to the patient a daily dose of peanut protein of about 4.0 mg for 52 weeks.
